(19) United States
(12) Patent Application Publication
(10) Pub. No.: US 2017/0290525 A1
(43) Pub. Date: Oct. 12, 2017

(54) ELECTROCARDIOGRAPH DEVICE AND METHOD

(71) Applicant: ENNOVEA MEDICAL, LLC, Columbus, OH (US)

(72) Inventors: Dan Waites, Cincinnati, OH (US); Robert Deans, Morgantown, IN (US); Peter Bielecki, Cleveland, OH (US)

(21) Appl. No.: 15/396,019

(22) Filed: Dec. 30, 2016

Related U.S. Application Data
(60) Provisional application No. 62/273,840, filed on Dec. 31, 2015.

(51) Int. Cl. A61B 5/0408; A61B 5/00

(52) U.S. Cl. CPC A61B 5/04085; A61B 5/6831; A61B 5/6823

(57) ABSTRACT

The exemplary embodiments herein provide an electrocardiograph device having a harness with a slot and an electrode comprising an electrode body, a proximate base extending away from the electrode body, and a conductive post extending away from the proximate base and located within the slot, said conductive post preferably having a shaft which connects to a distal tip. A retaining element is preferably located between the harness and the distal tip and slidably secures the conductive post within the slot.

### CROSS-REFERENCE TO RELATED APPLICATIONS

This application claims priority to US provisional application No. 62/273,840 filed on Dec. 31, 2015, which is herein incorporated by reference in its entirety.

### TECHNICAL FIELD

Embodiments generally relate to a device and method for placing electrodes when obtaining an ECG.

### BACKGROUND OF THE ART

An electrocardiograph is a diagnostic instrument widely used in the medical field where the electric pulses generated by an individual's heart are transformed by the electrocardiograph to a chart or graph, commonly referred to as an electrocardiogram, more commonly referred to as an ECG. This can provide a valuable tool for medical personnel to detect any abnormality in the individual's heart.

### SUMMARY OF THE EXEMPLARY EMBODIMENTS

In some situations, an ECG must be performed quickly and in every situation, it must be performed accurately. To accomplish both goals is a difficult task, especially when working under a stressful situation such as an emergency room or in the field at the site of an accident or catastrophe. Importantly, the electrodes of the electrocardiograph must be positioned properly in order to obtain the correct data for medical personnel. Further, it may be desirable to repeat the electrocardiograph process at a later date/time to determine efficacy of treatments or the patient's evolving condition. In this way, it is desirable to have a repeatable placement of the electrodes so that the medical personnel can accurately compare electrocardiograms from the same patient.

Exemplary embodiments provide an electrocardiographic device having a release liner, a plurality of electrodes, a harness, and a plurality of retaining elements. The release liner can be removed in portions, allowing any desired set of electrodes to be positioned within the harness and applied to the patient first. The remaining portions of the release liner can then be removed so that the remaining electrodes can be positioned within the harness and applied to the patient. Extensions of the release liner as well as voids and perforations within the release liner permit medical personnel to easily select and remove the desired portions. In some embodiments, the retaining elements and harness can be removed from the patient while leaving the electrodes in place for further ECGs at some point in the future.

The foregoing and other features and advantages of the present invention will be apparent from the following more detailed description of the particular embodiments, as illustrated in the accompanying drawings.

### BRIEF DESCRIPTION OF THE DRAWINGS

A better understanding of an exemplary embodiment will be obtained from a reading of the following detailed description and the accompanying drawings wherein identical reference characters refer to identical parts and in which:

FIG. 1 is a top plan view of an exemplary embodiment of the electrocardiograph device, indicating the location for section line A-A.

FIG. 2 is an exploded view of the embodiment shown in FIG. 1.

FIG. 3 is a top plan view of an exemplary embodiment of the release liner.

FIG. 4 is a top plan view of an exemplary embodiment of the release liner showing the locations for Detail views A, B, and C.

FIG. 5 is a detailed view of Detail A, shown in FIG. 4.

FIG. 6 is a detailed view of Detail B, shown in FIG. 4.

FIG. 7 is a detailed view of Detail C, shown in FIG. 4.

FIG. 8 is a top plan view of another exemplary embodiment of the harness.

FIG. 9 is another top plan view of an exemplary embodiment of the harness.

FIG. 10 is another top plan view of an exemplary embodiment of the harness.

FIG. 11 is a sectional view showing a section cut through section line A-A.

FIG. 12 is a flow chart for an exemplary embodiment of a method of using the electrocardiographic device shown and described herein.

### DETAILED DESCRIPTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/ or components, but do not preclude the presence or addition of one or more other features, integers, steps, operations, elements, components, and/or groups thereof.

Embodiments of the invention are described herein with reference to illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

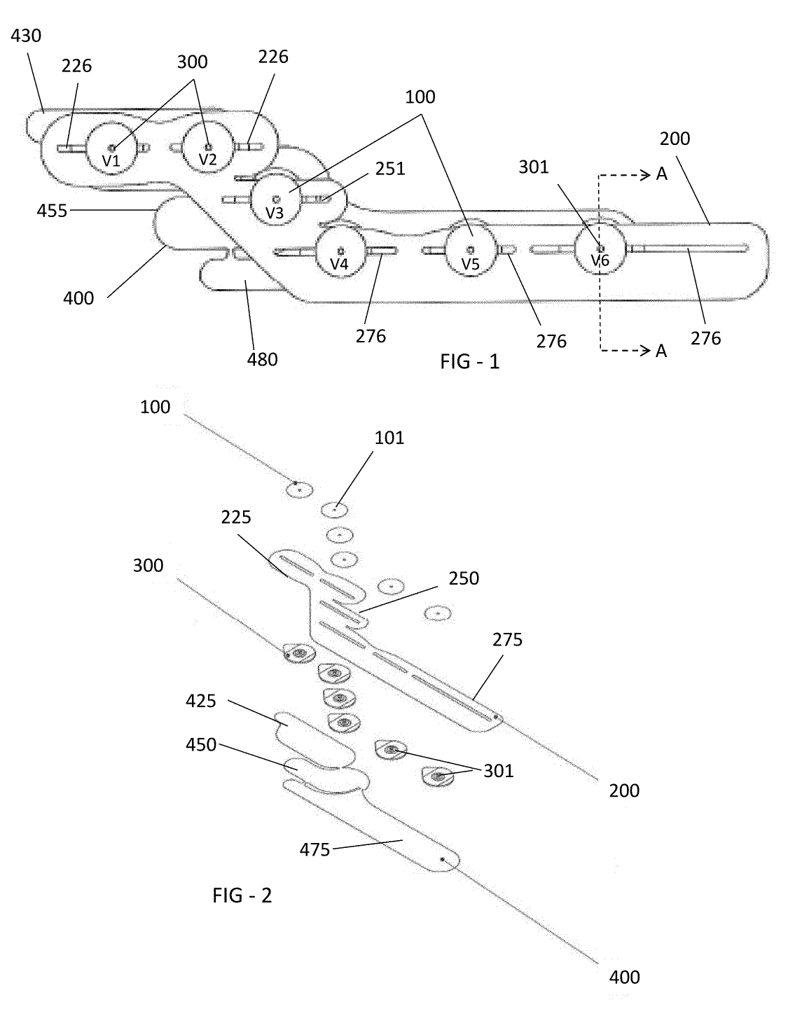

FIG. 1 is a top plan view of an exemplary embodiment of the electrocardiograph device. FIG. 2 is an exploded view of the embodiment shown in FIG. 1. This embodiment is generally comprised of four main layers of elements (from top to bottom): retaining elements 100, a harness 200, electrodes 300, and a release liner 400. The harness 200 can be described with three substantially parallel portions which are offset from one another, and each portion of the harness contains one or more slots for accepting one of the electrodes 300 designated V1-V6. The slots will be described further below.

The first portion 225 of the harness 200 contains slots 226 for the electrodes 300 designated as V1 and V2, which are generally placed over a patient's sternum. The second portion 250 contains slots 251 for the electrode 300 designated as V3, and generally connects with the first portion 25, preferably below the slot 226 for electrode 300 designated as V2. The third portion 275 contains slots 276 for the electrodes 300 designated V4-V6, and generally connects with the second portion 250, preferably below the slot 251 for electrode 300 designated as V3. The electrodes 300 designated as V4, V5, and V6 are preferably placed over the patient's mid-clavicular, anterior axillary, and mid-axillary areas of the chest respectively.

It is preferred that the slots 226 are aligned with each other and are generally parallel to slots 251 and 276. Similarly, it is preferable that slots 276 are aligned with one another and generally parallel to slots 226 and 251.

As shown, slot 251 designated for V3, overlaps vertically with the first portion 225 (and at least a portion of slot 226 designated for V1) as well as the third portion 275 (and at least a portion of the slot designated for V4). In other words, a portion of the slot 226 for V2 is directly above a portion of the slot 251 for V3, while a portion of the slot 251 for V3 is directly above a portion of the slot 276 for V4. It can also be said that slot 251 contains a left end which is located vertically below the midpoint of slot 226 for V2, and also contains a right end which is located vertically above the midpoint of slot 276 for V4.

Each electrode 300 preferably contains a rear surface containing an adhesive 302 and a front surface containing a conductive post 301 (typically metallic or radiolucent carbon fiber). See FIG. 11 below for details. To secure the electrodes 300 within their designated slots within the harness 200, the conductive post 301 may be inserted into the back of the harness and through the designated slot so that an opening within the retaining element 100 can accept the conductive post 301. This will secure the electrode 300 within the slot of the harness 200 while still allowing the electrode 300 to slide within the slot. It should be noted that while a disc or circular shape is shown for the retaining elements 100 this is not required, as any shape would work with the various embodiments. It is also not necessary for each retaining element 100 to have the same shape, as some could be circular while others are rectangular, and some may be circular with a smaller diameter while others are circular with a larger diameter.

Each retaining element 100 preferably contains an opening 101 sized to accept a portion of the conductive post 301 in order to slidably secure the electrode 300 to the harness 200 (i.e. the electrode 300 is secured within the harness 200 but is permitted to slide within its designated slot). Generally, a small interference fit between the opening 101 within the retaining element 100 and a portion of the conductive post 301 may be used to hold the electrode 300 within the slot, while still permitting it to slide within the slot.

The release liner 400 is also generally comprised of three portions 425, 450, and 475 which align with the three portions 225, 250, and 275 of the harness 200 respectively generally, but not exactly. As shown in FIG. 1, an extension 430 of the first portion 425 of the release liner 400 extends away from and outside of the boundaries of the first portion 225 of the harness 200. In this embodiment the extension 430 extends to the left of the edge of the harness 200, but it could extend above or to the right as well. Similarly, an extension 455 of the second portion 450 of the release liner 400 extends away from and outside of the boundaries of the second portion 250 of the harness 200. Here, the extension 455 extends to the left. Finally, an extension 480 of the third portion 475 of the release liner extends away from and outside of the boundaries of the third portion 275 of the harness 200. Here, the extension 480 extends to the left.

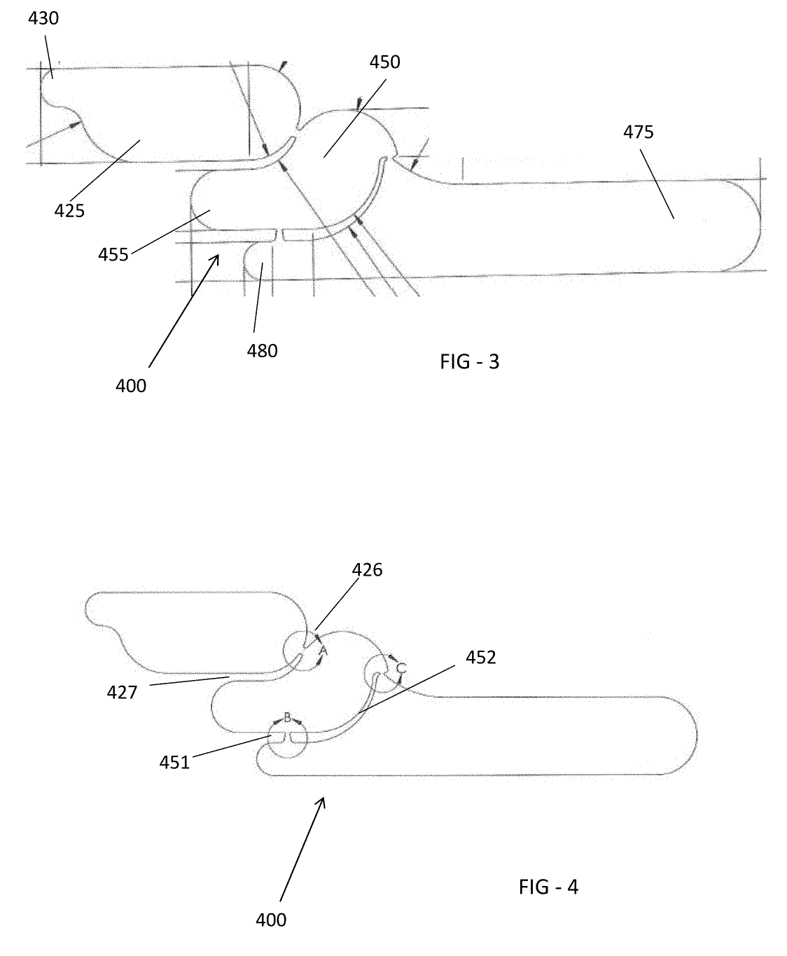

FIG. 3 is a top plan view of an exemplary embodiment of the release liner 400. FIG. 4 is a top plan view of an exemplary embodiment of the release liner 400 showing the locations for Detail views A, B, and C. Also shown here is the lower void 427 between the first portion 425 and second portion 450 as well as the upper void 426 between the first portion 425 and the second portion 450. A lower void 451 is shown between the second portion 450 and the third portion 475 along with an upper void 452 also shown between the second portion 450 and the third portion 475.

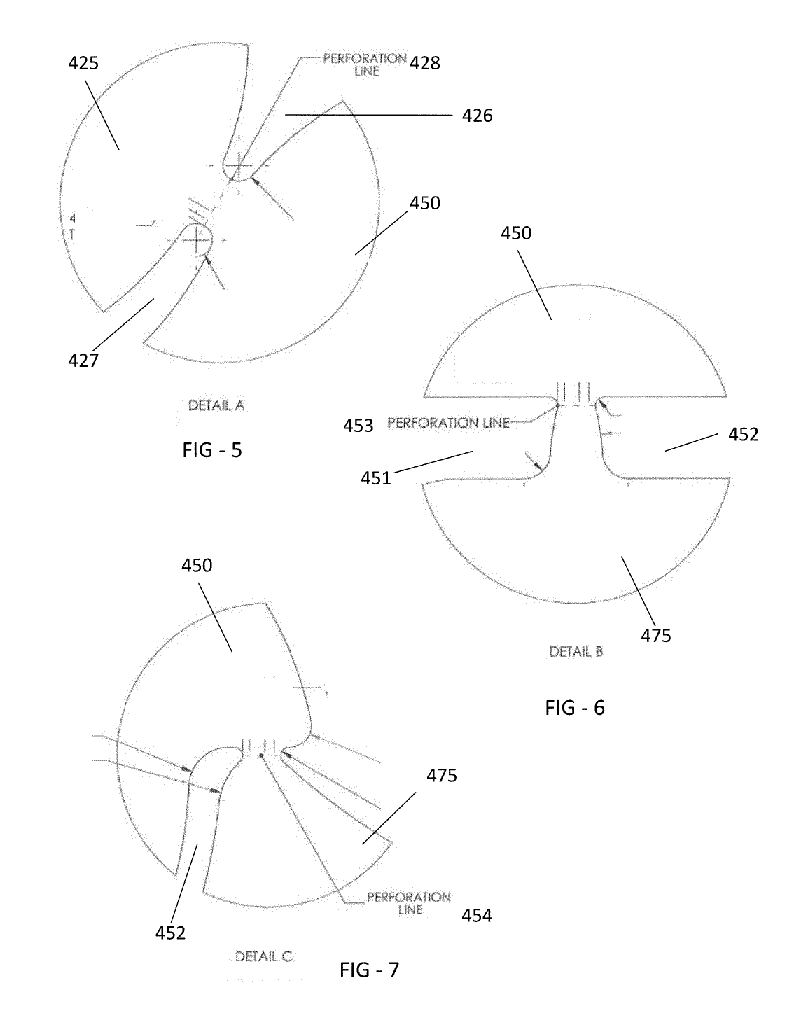

FIG. 5 is a detailed view of Detail A, shown in FIG. 4. As mentioned above, the lower void 427 is preferably located between the first portion 425 and second portion 450 as well as an upper void 426 which is preferably located between the first portion 425 and the second portion 450. A perforation line 428 may define the general intersection between the first portion 425 and second portion 450 and is generally aligned with the curve defined by the voids 426 and 427. The perforation line 428 should allow for relatively easy mechanical separation of the first portion 425 from the second portion 450 or vice versa.

FIG. 6 is a detailed view of Detail B, shown in FIG. 4. As mentioned above, a lower void 451 is shown between the second portion 450 and the third portion 475 along with an upper void 452 also shown between the second portion 450 and the third portion 475. A perforation line 453 may define the general intersection between the second portion 450 and the third portion 475 and is generally aligned with the curve defined by the voids 451 and 452. The optional perforation line 453 should allow for relatively easy mechanical separation of the second portion 450 from the third portion 475 or vice versa.

FIG. 7 is a detailed view of Detail C, shown in FIG. 4. Here we see the upper void 452 ending at the location of the perforation line 453 which may define the general intersection between the second portion 450 and the third portion 475. The optional perforation line 453 is generally located at the perimeter of the release liner 400, between the second portion 450 and the third portion 475. The perforation line 454 should allow for relatively easy mechanical separation of the second portion 450 from the third portion 475 or vice versa.

In an exemplary embodiment, the first portion 425 and the second portion 450 may be separated during manufacturing while the second portion 450 and the third portion 475 would be separated prior to placing the initial portion on a female patient.

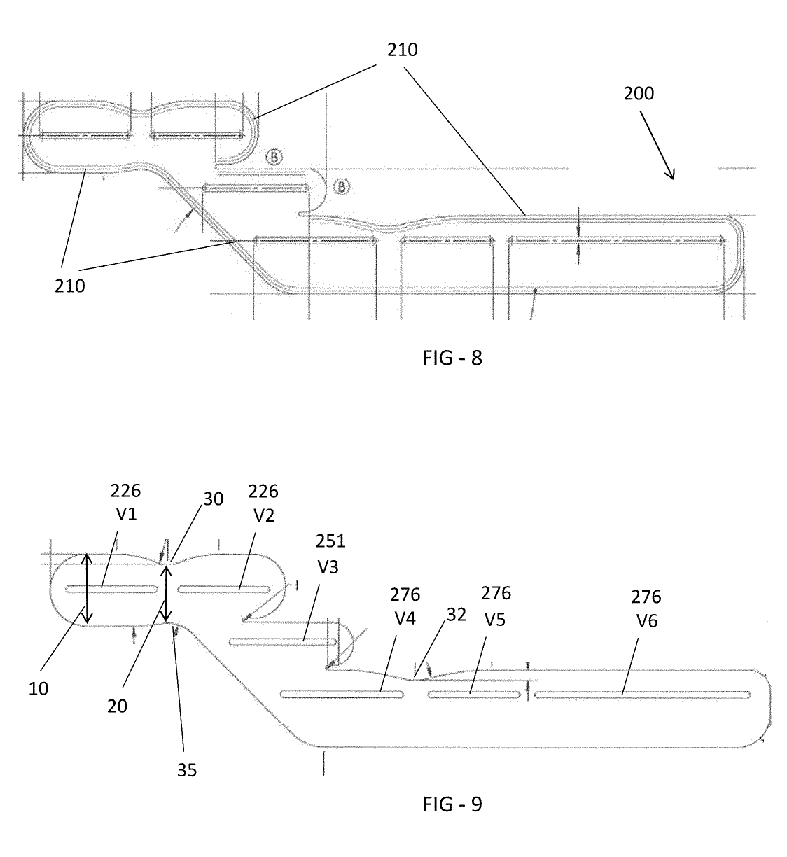

FIG. 8 is a top plan view of another exemplary embodiment of the harness 200. In this embodiment, the perimeter 210 of the harness 200 contains a kiss cut or is otherwise pressed around the perimeter 210 in order to compress the layers of the harness 200 without actually cutting them. This process has been discovered to produce rolled edges of the harness 200 to make them more flexible.

FIG. 9 is another top plan view of an exemplary embodiment of the harness 200. Here, the slots 226, 251, and 276 within the harness are clearly shown, which correspond to the electrodes 300 designated V1-V6 described above. Also shown here is the optional reduced width of the harness 200 around the sternum area of the patient and found between slots 226 for the V1 and V2 electrodes 300. As shown, the first portion of the harness 225 generally has a thickness 10, but this is reduced to a smaller thickness 20 at this point between slots 226 for the V1 and V2 electrodes 300. In other words, a first notch 30 may be placed on the top side of the first portion 225 of the harness while a second notch 35 may be placed on the bottom side of the first portion 225 of the harness, where both notches 30/35 are placed between slots 226 for the V1 and V2 electrodes 300 and are preferably aligned vertically with one another. The two notches 30/35 may therefore combine to reduce the overall vertical height of the first portion of the harness 225.

Similarly, a notch 32 may also be placed on the top side of the third portion 275 of the harness 200 and located between slots 276 designated for V4 and V5.

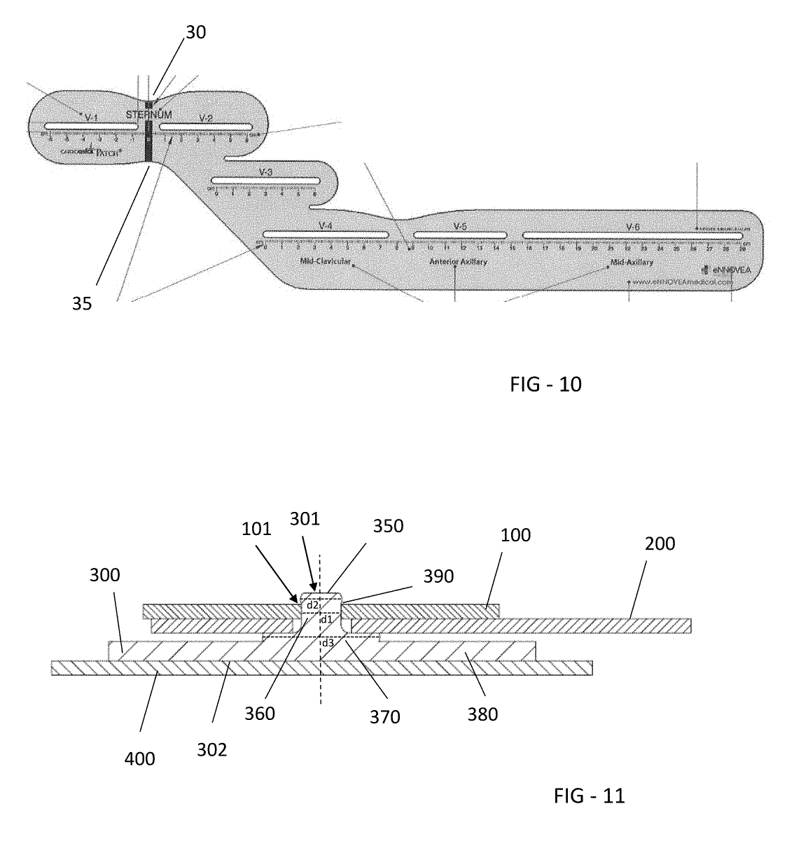

FIG. 10 is another top plan view of an exemplary embodiment of the harness. As shown, a scale, ruler, or other measurement device may be overlaid on top of the harness 200 and across the slots 226, 251, and 276 in order to ensure accurate and repeatable placement of the electrodes 300 as well as accurate record keeping by the medical personnel.

FIG. 11 is a sectional view showing a section cut through section line A-A, which passes through the conductive post 31 of an electrode 300. As noted above, each electrode 300 preferably contains a rear surface containing an adhesive 302 and a front surface containing a conductive post 301 (typically metallic or radiolucent carbon fiber). The release liner 400 is adhered to the rear surface of the electrode 300 using a layer of adhesive 302.

The electrode 300 preferably contains several different portions, and begins with the electrode body 380, which as noted above preferably contains a layer of adhesive 302 on the back side for temporarily adhering to a patient for one or more procedures. A proximal base 370 extends above the electrode body 380 and contains a top surface. Generally speaking, the proximal base 370 is preferably symmetrical about the center axis of the electrode 300. The conductive post 301 extends upwardly from the proximal base 370 and preferably contains a shaft 360 which connects to a distal tip 350. It is preferable that the shaft 360 contain a first diameter d1, while the distal tip 350 contains a second diameter d2, where the first diameter d1 is smaller than the second diameter d2. It is also preferable that the third diameter d3 for the proximal base 370 is larger than both d1 and d2.

Since the electrode 300 shown in this embodiment is generally circular and/or symmetrical about a central axis, d1-d3 are referred to as diameters, but it should be recognized by any person having skill in the art that electrodes 300 in other embodiments could be rectangular or oval shaped, such that d1-d3 could simply be dimensions d1-d3 rather than diameters d1-d3.

The change in diameter from the smaller diameter d1 to the larger diameter d2 ono the conductive post 301 creates a ledge 390 where the shaft 360 transitions to the distal tip 350. This ledge 390 may contact the front (or top) surface of the retaining element 100, to prevent the conductive post 301 from being removed from the designated slot in the harness 200, while still permitting the conductive post 301 to slide within the slot and relative to the harness 200. As noted above, the retaining element 100 preferably contains an opening 101 for accepting the conductive post 301 and it is preferred that the size of the opening 101 is slightly smaller than diameter d2, to further prevent the distal tip 350 from passing through the retaining element 100 and allowing the conductive post 301 to be removed from the harness 200. When the device is fully assembled, the retaining element 100 and harness 200 should be sandwiched in between the ledge 390 of the distal tip 350 and the top surface of the proximal base 370. The retaining element 100 can be comprised of any number of flexible or rigid materials, including but not limited to plastics, paper, and composites.

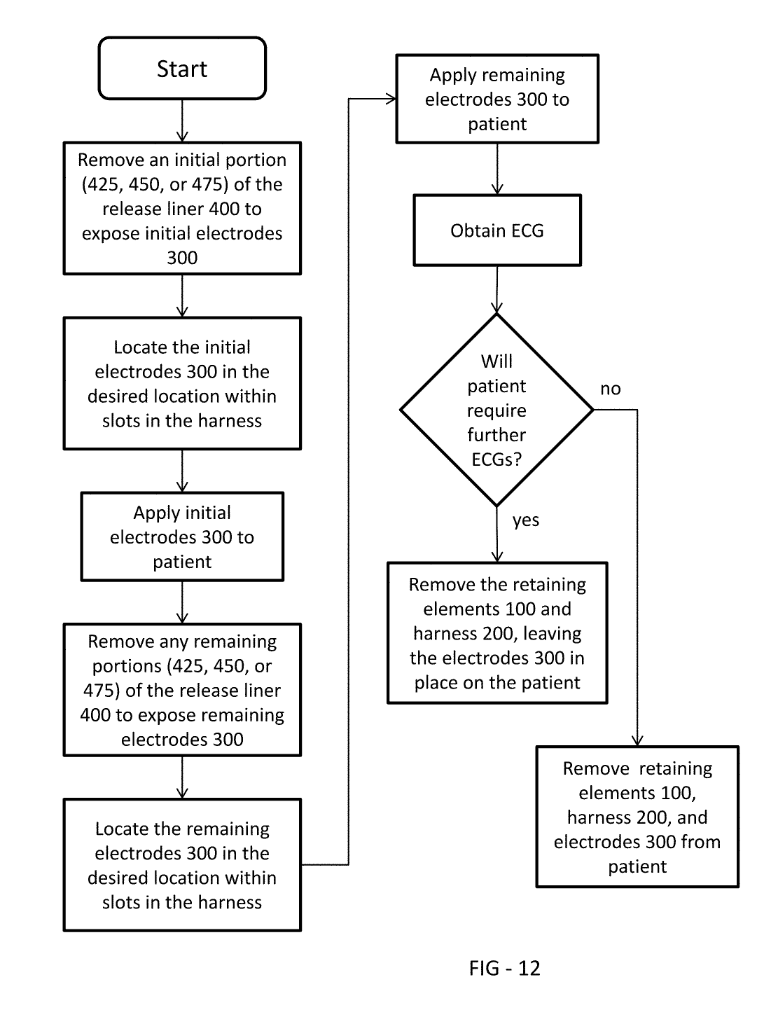

FIG. 12 is a flow chart for an exemplary embodiment of a method of using the electrocardiographic device shown and described herein. Initially, as described above, the three portions (425, 450, and 475) of the release liner 400 can be mechanically separated along the perforation lines. In this way, only a portion of the release liner 400 may be removed at a time, to allow for specific electrodes 300 to be placed on the patient first. It has been found that the precise placement procedure can vary depending on the patient, and specifically whether the patient is a male or female.

Once the initial portion of the release liner 400 is removed, the adhesive on the back of the initial electrodes 300 (i.e. those contained within the initial portion removed) is now exposed, and the corresponding electrodes 300 (i.e. those that have had their portion of the release liner 425, 450, and 475 removed) may be placed within their desired location within slots 226, 251, and/or 276. Once located, the initial electrodes 300 may be placed on the patient.

Now, any remaining portions (425, 450, and 475) of the release liner 400 may be removed, so that the remaining electrodes 300 can be located within slots 226, 251, and/or 276, and applied to the patient. At this point the ECG can be obtained (pending any other intermediary steps such as connecting the conductive posts 301 of the electrodes 300 to diagnostic equipment). If further ECGs will be necessary with this patient, the medical personnel has the option of removing the retaining elements 100 so that the harness 200 can be removed while leaving the electrodes 300 in their precise location on the patient. This allows for subsequent ECGs to be obtained while using the same locations for the electrodes 300 (and prevents the waste of having to discard the first set of electrodes 300). If however no further ECGs will be required by this patient, the retaining elements 100, harness 200, and electrodes 300 may be removed from the patient.

Having shown and described a preferred embodiment of the invention, those skilled in the art will realize that many variations and modifications may be made to affect the described invention and still be within the scope of the claimed invention. Additionally, many of the elements indicated above may be altered or replaced by different elements which will provide the same result and fall within the spirit of the claimed invention. It is the intention, therefore, to limit the invention only as indicated by the scope of the claims.

We claim:

1. An electrocardiograph device comprising:
a harness having a slot;
an electrode comprising:
an electrode body,
a proximate base extending away from the electrode body, and
a conductive post extending away from the proximate base and located within the slot, said conductive post having a shaft which connects to a distal tip; and
a retaining element which is located between the harness and the distal tip.

2. The electrocardiograph device of claim 1 wherein:
the electrode body, conductive post, and retaining element are substantially symmetrical about a central axis.

3. The electrocardiograph device of claim 1 wherein:
the harness and retaining element are positioned in between the distal tip and the proximate base.

4. The electrocardiograph device of claim 1 wherein:
the shaft has a diameter d1,
the distal tip has a diameter d2, and
d2 is greater than d1.

5. The electrocardiograph device of claim 1 wherein:
the proximate base has a diameter d3,
the shaft has a diameter d1, and
d3 is greater than d1.

6. The electrocardiograph device of claim 1 wherein:
the shaft has a diameter d1,
the distal tip has a diameter d2,
the proximate base has a diameter d3, and d1<d2<d3.

7. The electrocardiograph device of claim 1 wherein:
the retaining device contains an opening which is sized to accept the conductive post.

8. The electrocardiograph device of claim 7 wherein:
the distal tip has a diameter of d2 and
the opening within the retaining device is smaller than d2.

9. An electrocardiograph device comprising:
a harness having a slot;
an electrode comprising
an electrode body and
a conductive post extending away from a proximate base and located within the slot; and
a retaining element having an opening which is sized to accept the conductive post and is positioned atop the harness.

10. The electrocardiograph device of claim 9 further comprising:
a kiss cut around a perimeter of the harness.

11. The electrocardiograph device of claim 9 wherein:
the conductive post includes a distal tip.

12. The electrocardiograph device of claim 11 wherein:
the distal tip has a diameter d2 where the opening in the retaining element is smaller than d2.

13. The electrocardiograph device of claim 9 further comprising:
adhesive placed on the electrode; and
a release liner placed on the adhesive.

14. The electrocardiograph device of claim 13 wherein:
the release liner secures the conductive post within the slot, but once the release liner is removed the conductive post is free to slide within the slot.

15. The electrocardiograph device of claim 13 further comprising:
a second slot in the harness;
a second electrode comprising
an electrode body and
a conductive post extending away from the proximate base and located within the second slot;
a second retaining element having an opening which is sized to accept the conductive post and is positioned atop the harness;
adhesive placed on the second electrode;
a second release liner placed on the adhesive for the second electrode;
where the release liner and second release liner are connected at a perforation line.

16. An electrocardiograph device comprising:
a harness having a slot and a perimeter;
a kiss cut around the perimeter of the harness;
an electrode having a conductive post; and
a retaining element positioned around the conductive post to secure the conductive post within the slot while still permitting the conductive post to slide within the slot.

17. The electrocardiograph device of claim 16 wherein:
the retaining element is placed atop the harness.

18. The electrocardiograph device of claim 16 wherein:
the conductive post comprises a shaft with a distal tip, where the distal tip has a larger diameter than the shaft.

19. The electrocardiograph device of claim 18 wherein:
the retaining element is located below the distal tip of the conductive post.

20. The electrocardiograph device of claim 18 further comprising:
a proximal base extending from the electrode body and connecting with the shaft of the conductive post, and
where the harness and retaining element are positioned in between the proximal base and the distal tip of the conductive post.
